(19) United States
(12) Patent Application Publication
(10) Pub. No.: US 2010/0007979 A1
(43) Pub. Date: Jan. 14, 2010

(54) MAGNETIC DISC APPARATUS, GAS SENSOR, AND THEIR MANUFACTURE METHOD

(75) Inventor: Mineharu Tsukada, Kawasaki (JP)

Correspondence Address: GREER, BURNS & CRAIN, 300 S WACKER DR, 25TH FLOOR, CHICAGO, IL, 60606, US

(73) Assignee: FUJITSU LIMITED, Kawasaki-shi (JP)

(21) Appl. No.: 12/484,809

(22) Filed: Jun. 15, 2009

(30) Foreign Application Priority Data
Jul. 9, 2008 (JP) 2008-178876

(51) Int. Cl. G11B 27/36; G01N 33/00; B05D 5/12

(52) U.S. Cl. 360/31; 73/31.05; 427/77; G9B/27.052

(57) ABSTRACT

A magnetic recording medium having a first lubricant layer formed on the surface thereof and a gas sensor are disposed in a housing. The gas sensor detects gas by adsorbing the gas on a detection surface thereof The detection surface is formed with a second lubricant layer made of the same lubricant agent as that of the first lubricant layer.

### CROSS-REFERENCE TO RELATED APPLICATION

This application is based upon and claims the benefit of priority of the prior Japanese Patent Application No. 2008-178876, filed on Jul. 9, 2008, the entire contents of which are incorporated herein by reference.

### FIELD

The embodiments discussed herein are related to a magnetic disc apparatus having a magnetic recording medium and a gas sensor built therein, a gas sensor, and a manufacture method for a magnetic recording medium and a gas sensor.

### BACKGROUND

While a magnetic disc apparatus is driven, gas generated in a housing of the apparatus or gas invaded from the outside of the housing adsorbs onto the surfaces of a magnetic recording medium or a magnetic head. The adsorbed gas adversely affects reliability of an Head Disc Interface (HDI). It is possible to predict beforehand a trouble of a magnetic disc apparatus by monitoring a change in a gas amount in the housing (Japanese Laid-open Patent Publication No. 2007-35180). A quartz crystal micro balance sensor (QCM sensor) may be used for monitoring a gas amount. The QCM sensor detects a mass change of a surface of a quartz oscillator and measures a frequency.

### SUMMARY

As gas adsorbs onto the surface of an electrode surface of a QCM sensor, it is possible to detect the gas. It is therefore difficult to detect gas belonging to gaseous species that is easy to adsorb onto the surface of the magnetic disc but difficult to adsorb onto the electrode surface of a QCM sensor. Conversely, a detection sensitivity is extremely high for gas belonging to gaseous species that is difficult to adsorb onto the surface of a magnetic disc but easy to adsorb onto the electrode surface of a QCM sensor.

According to an aspect of the invention, a magnetic disc apparatus including:

a magnetic recording medium disposed in a housing and having a first lubricant layer formed on a surface of the magnetic recording medium; and

a gas sensor disposed in the housing for detecting gas by adsorbing the gas on a detection surface of the gas sensor, the detection surface being formed with a second lubricant layer made of a same lubricant agent as a lubricant agent used for the first lubricant layer.

According to another aspect of the invention, a gas sensor including:

a substrate made of piezoelectric material;

a first electrode formed over a first surface of the substrate;

a second electrode formed on a second surface of the substrate other than the first surface; and

a lubricant layer made of a lubricant agent and formed on the first electrode.

According to still another aspect of the invention, a manufacture method for a magnetic recording medium and a gas sensor, including:

disposing a first substrate made of non-magnetic material and a second substrate made of piezoelectric material in a same film forming chamber;

forming a magnetic layer over the first substrate and forming a magnetic layer made of a same magnetic material as a magnetic material of the magnetic layer and used as an electrode, respectively in the film forming chamber;

forming a protective layer made of non-magnetic material on the magnetic layer and forming a protective layer made of a same non-magnetic material as the non-magnetic material of the protective layer on the electrode, respectively in the film forming chamber;

unloading the first substrate formed with the protective layer and the second substrate formed with the protective layer, respectively from the film forming chamber;

forming a lubricant layer on the protective layer of the first substrate; and

forming a lubricant layer made of a same lubricant agent as a lubricant agent of the lubricant layer formed on the first substrate, on the protective layer of the second substrate.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a plan view illustrating the inside of a housing of a magnetic disc apparatus of a first embodiment.

FIGS. 2A and 2B are a plan view and a cross sectional view illustrating a gas sensor of the first embodiment.

FIG. 3 is a cross sectional view illustrating a gas sensor of a second embodiment.

FIG. 4A is a cross sectional view illustrating a gas sensor of a third embodiment during manufacture, and FIG. 4B is a cross sectional view illustrating the gas sensor of the third embodiment.

FIG. 5 is a cross sectional view illustrating a gas sensor of a fourth embodiment.

FIG. 6 is a cross sectional view illustrating a gas sensor of a fifth embodiment.

FIGS. 7A to 7F are cross sectional views illustrating the gas sensor and a magnetic disc of the fifth embodiment during manufacture, and FIG. 7G is a cross sectional view illustrating the gas sensor and the magnetic disc of the fifth embodiment.

### DESCRIPTION OF EMBODIMENTS

The first to fifth embodiments will now be described with reference to the accompanying drawings.

### [a] First Embodiment

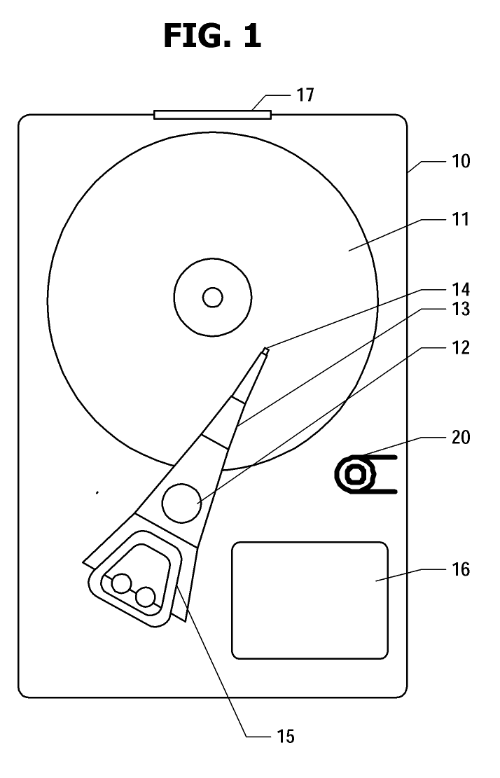

FIG. 1 is a plan view illustrating the inside of a housing of a magnetic disc apparatus of the first embodiment. A magnetic disc (magnetic recording medium) 11 is accommodated in the housing 10. The magnetic disc 11 rotates around its center. A magnetic head 14 is mounted at a distal end of an arm 13. The magnetic head 14 is supported above a surface of the magnetic disc 11. An arm driving magnet 15 is mounted at a rear end portion of the arm 13. The arm driving magnet 15 swings the arm 13 around a supporting point 12. As the arm 13 swings, the magnetic head 14 moves in the radial direction of the magnetic disc 11. A control circuit 16 controls rotation of the magnetic disc 11, drive of the arm 13, the magnetic head 14, and other operations.

The housing 10 is provided with a window 17 with a filter. Gas is transported from the inside to outside or from the outside to inside of the housing 10, via the window 17 with a filer. A gas sensor 20 is disposed in the housing 10. The gas sensor 20 detects gas in the housing 10. For example, a QCM sensor is used as the gas sensor 20. As the magnetic disc 11 rotates, an air current is generated. The gas sensor 20 is disposed at a position which the air current generated by the rotation of the magnetic disc 11.

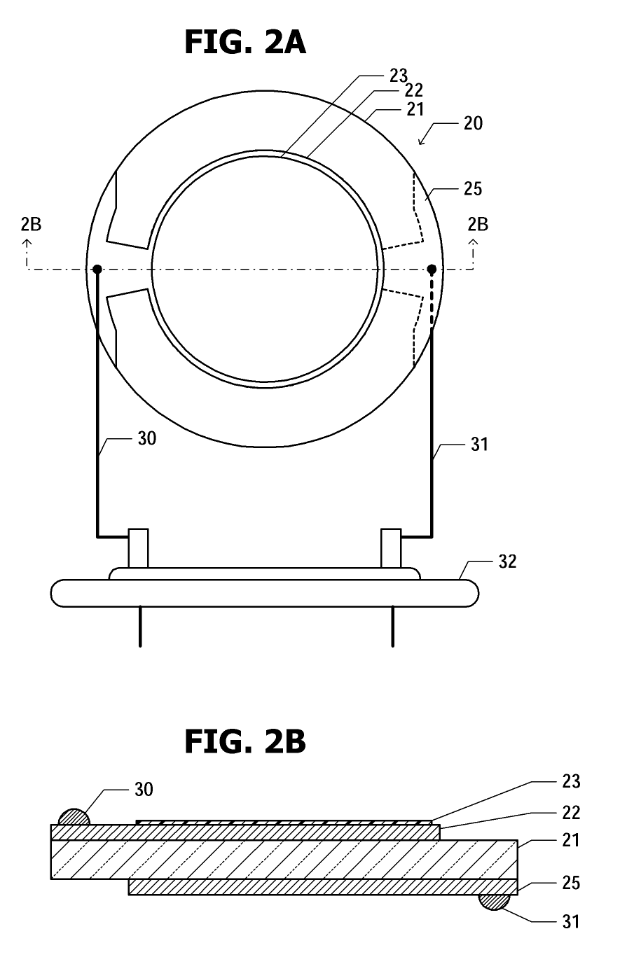

FIG. 2A is a plan view of the gas sensor 20, and FIG. 2B is a cross sectional view taken along one-dot chain line 2B-2B in FIG. 2A.

On one surface of a substrate 21 of a disc shape made of piezoelectric material, a first electrode 22 is formed, and a second electrode 25 is formed on the other surface. As the substrate 21, for example, an AT cut crystal substrate having a thickness of about 0.5 mm is used. Substrates made of other piezoelectric materials may also be used. The first electrode 22 and the second electrode 25 are made of, for example, gold (Au). Thickness of each of the first electrodes 22 and the second electrode 25 is, for example, 100 nm.

Each of the first electrode 22 and the second electrode 25 includes a circular potion concentric with the substrate 21 and a lead wire connection portion extending from the circular portion toward the border of the substrate 21. As viewed in plan, the circular portion of the first electrode 22 and the circular portion of the second electrode 25 overlap with each other. The lead wire connection portion of the first electrode 22 and the lead wire connection portion of the second electrode 25 mutually extend toward the opposite direction.

The first electrode 22 and the second electrode 25 may be formed by covering the area, where the electrodes are not formed, by a mask, depositing an Au film by sputtering, and thereafter removing the mask together with the Au film deposited on the mask. The first and second electrodes 22, 25 may also be formed by forming an Au film on the whole surfaces of the substrate 21 and then patterning the Au film.

One end of a first lead wire 30 and one end of a second lead wire 31 are connected to the lead wire connection portions of the first electrode 22 and the second electrode 25, respectively. The first lead wire 30 and the second lead wire 31 have a mechanical strength sufficient for supporting the substrate 21. The other ends of the first lead wire 30 and the second lead wire 31 are fixed to a base 32.

Lubricant agent is coated on the surface of the circular portion of the first electrode 22 to form a lubricant layer 23. A thickness of the lubricant layer 23 is, for example, in the range of 1 nm to 2 nm. The surface of the magnetic disc 11 illustrated in FIG. 1 is generally formed with a protective layer of diamond like carbon (DLC), and the surface of the protective layer is further covered with a lubricant layer. The lubricant layer 23 formed on the surface of the first electrode 22 is made of the same lubricant agent as that used for the lubricant layer formed on the surface of the magnetic disc 11.

The lubricant layer 23 is formed, for example, by dropping lubricant agent onto the surface of the first electrode 22. The dropped lubricant agent diffuses to the region near the border of the first electrode 22 to form the circular lubricant layer 23 covering almost the whole surface of the first electrode 22. The lubricant layer 23 may protrude from the first electrode 22 and cover the exposed surface of the substrate 21.

The lubricant layer 23 covering the surface of the first electrode 22 has the same gas adsorption ability as that of the lubricant layer formed on the surface of the magnetic disc 11. Therefore, gas that actually adsorbs onto the surface of the magnetic disc 11 is able to be detected with the gas sensor 20. It is therefore possible to detect at a high precision an adsorption state of gas on the surface of the magnetic disc 11. It is also possible to avoid excessive reaction by gas difficult to adsorb onto the surface of the magnetic disc 11.

### [b] Second Embodiment

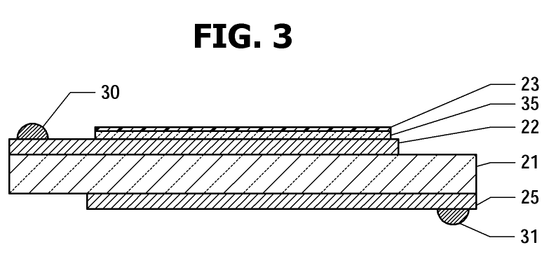

FIG. 3 is a cross sectional view of a gas sensor of the second embodiment. In the second embodiment, a protective layer 35 made of DLC is disposed between the first electrode 22 and the lubricant layer 23. A thickness of the protective layer 35 is, for example, in a range of 4 nm to 5 nm.

Description will now be made on a method of forming the protective layer 35. First, an area where the protective film 35 is not formed is covered with a mask such as a resist pattern. In the state that the mask is formed, a DLC film is formed by sputtering or chemical vapor deposition (CVD). The mask is thereafter removed to leave the patterned protective layer 35.

Adhesion performance of the lubricant layer 23 is able to be improved by intervening the protective layer 35 between the first electrode 22 and the lubricant layer 23.

### [c] Third Embodiment

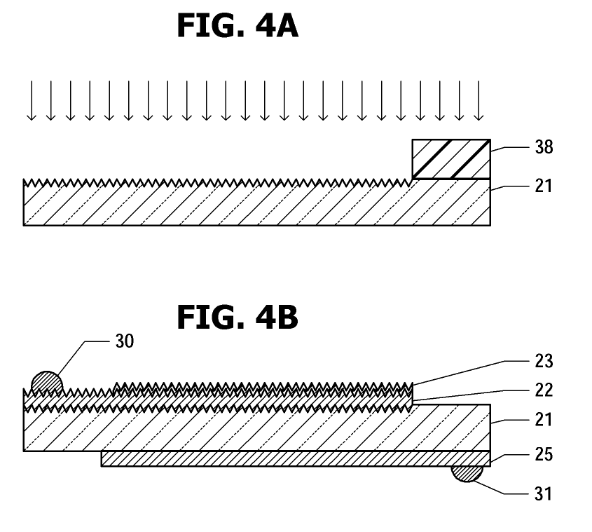

A gas sensor and its manufacture method of the third embodiment will be described with reference to FIGS. 4A and 4B.

As illustrated in FIG. 4A, a resist pattern 38 is formed on the surface of a substrate 21 made of piezoelectric material. The resist pattern 38 covers the area where the first electrode 22 illustrated in FIG. 2A is not formed. By using the resist pattern 38 as a mask, the surface of the substrate 21 is made rough by sand blast. The surface may be made rough by etching with hydrofluoric acid.

As illustrated in FIG. 4B, a first electrode 22 is formed on the roughed surface of the substrate 21. The surface of the first electrode 22 has asperity inheriting the surface condition of the underlying layer. A first lead wire 30 is connected to the lead wire connection portion of the first electrode 22. The circular portion of the first electrode 22 is covered with a lubricant layer 23.

A second electrode 25 is formed on the back surface of the substrate 21. A second lead wire 31 is connected to the second electrode 25.

In the third embodiment, since the surface of the underlying layer of the lubricant layer 23 is made rough, the lubricant layer 23 may have a larger surface area. It is therefore possible to increase a gas detection sensitivity. Also in the third embodiment, similar to the second embodiment illustrated in FIG. 3, a protective layer 35 of DLC may be disposed between the first electrode 22 and the lubricant layer 23.

### [d] Fourth Embodiment

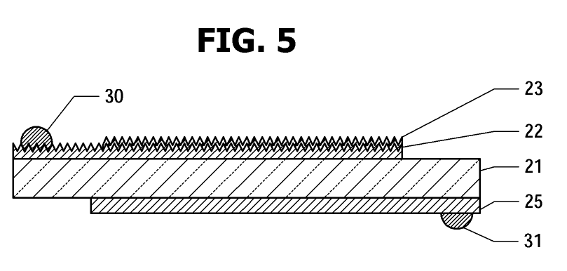

FIG. 5 is a cross sectional view of a gas sensor of the fourth embodiment. In the embodiment illustrated in FIG. 4B, the surface of the substrate 21 is subjected to a roughing process. In contract, in the fourth embodiment, the surface of the first electrode 22 is subjected to a roughing process. It is possible to obtain the same advantages as those of the third embodiment also by making the surface of the first electrode 22 be subjected to a roughing process.

### [e] Fifth Embodiment

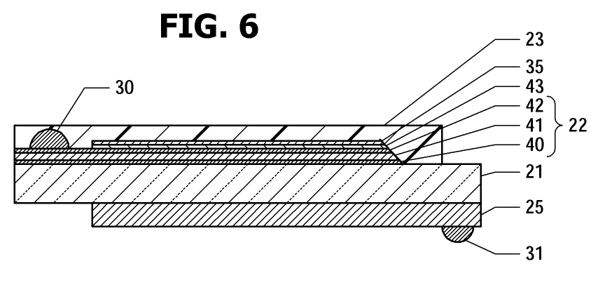

FIG. 6 is a cross sectional view of a gas sensor of the fifth embodiment. In the fifth embodiment, the first electrode 22 includes an underlying layer 40, a soft magnetic liner 41 and an intermediate layer 42. These layers 40 to 42 are made of magnetic material.

A magnetic recording layer 43 of CoCrPt—SiO₂ having a granular structure is formed on the circular portion of the first electrode 22. A protective layer 35 is formed on the magnetic recording layer 43. A first lead wire 30 is connected to the lead wire connection portion of the first electrode 22. A lubricant layer 23 covers the protective layer 35 and the lead wire connection portion of the first electrode 22.

A second electrode 25 is formed on the back surface of the substrate 21. A second lead wire 31 is connected to the second electrode 25.

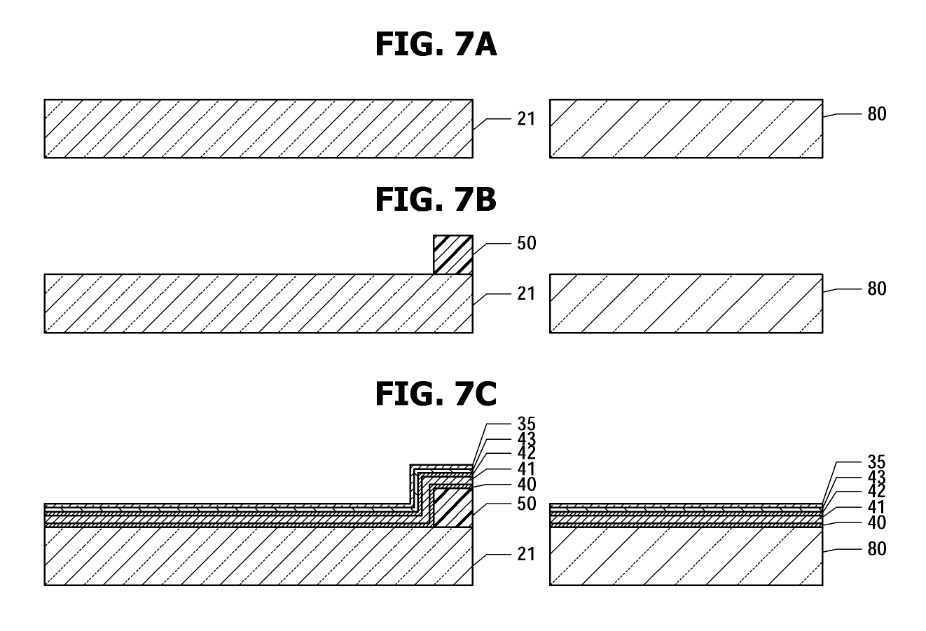

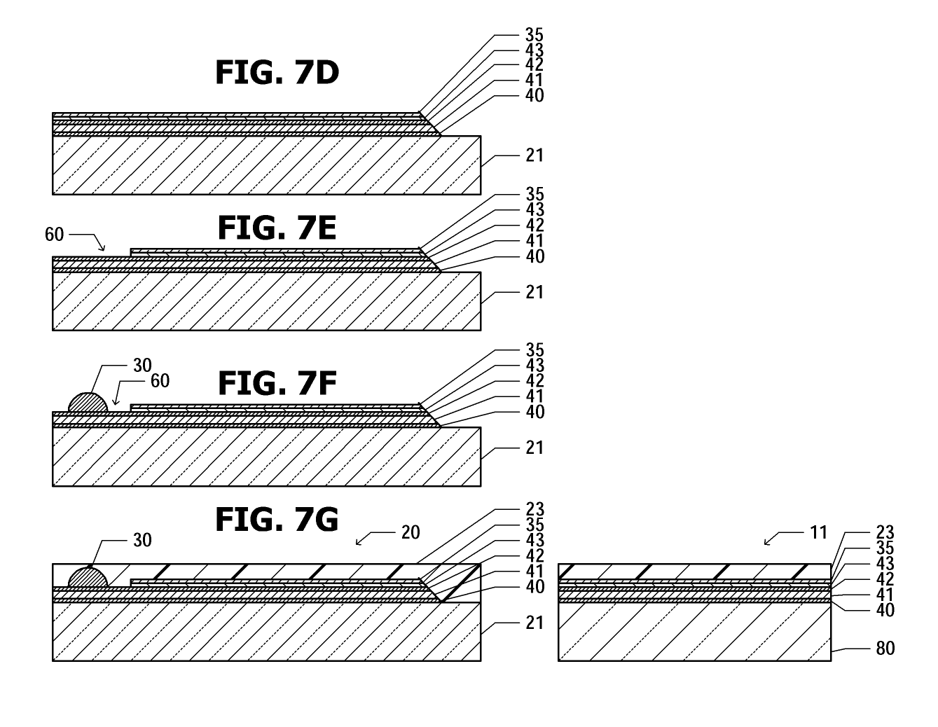

A manufacture method for the gas sensor of the fifth embodiment will be described with reference to FIGS. 7A to 7G.

FIG. 7A illustrates a substrate 21 for a gas sensor, and a substrate made of non-magnetic material for a magnetic disc, e.g., a glass substrate 80. As illustrated in FIG. 7B, a resist pattern 50 is formed on the substrate 21. The resist pattern 50 covers the area where the first electrode 22 illustrated in FIG. 6 is not formed. The substrate 21 formed with the resist pattern 50 and the glass substrate 80 are loaded in the same film forming chamber.

As illustrated in FIG. 7C, on the substrate 21 and the glass substrate 80, an underlying layer 40, a soft magnetic liner 41, an intermediate layer 42 and a magnetic recording layer 43 are formed, for example, by sputtering. For example, the underlying layer 40 is made of Cr, and has a thickness of 5 nm. The soft magnetic liner 41, for example, includes three layers of CoNbZr, Ru and CoNbZr, these three layers being stacked in this order, and has a total thickness of 150 nm. The intermediate layer 42, for example, includes three layers of Ta, NiFe and Ru, these three layers being stacked in this order, and has a total thickness of 30 nm. For example, the magnetic recording layer 43 has a granular structure made of CoCrPt—SiO₂, and has a thickness of 20 nm.

The substrate 21 and the glass substrate 80 formed with the layers up to the magnetic recording layer 43 are transported from the film forming chamber for sputtering to a film forming chamber for CVD. A protective layer 35 of non-magnetic material, e.g., DLC is formed on the magnetic recording layer 43 by CVD. A thickness of the protective layer 35 is, for example, 4 nm. After the protective layer 35 is formed, the substrate 21 and glass substrate 80 are unloaded from the film forming chamber.

As illustrated in FIG. 7D, the resist pattern 50 on the substrate 21 is removed together with each layer deposited on the resist pattern 50. In this manner, each layer from the underlying layer 40 to the protective layer 35 is patterned in the same flat plan shape as that of the first electrode 22.

As illustrated in FIG. 7E, two layers of the protective layer 35 and the magnetic recording layer 43 are patterned to expose the intermediate layer 42 in an area 60 corresponding to the lead wire connection portion of the first electrode 22.

As illustrated in FIG. 7F, a first lead wire 30 is connected to the area 60 exposing the intermediate layer 42.

As illustrated in FIG. 7G, a lubricant layer 23 is formed on the substrate 21. The lubricant layer 23 covers the protective layer 35. Up to these processes, the gas sensor 20 is completed. A single large substrate 21, on which a plurality of gas sensors 20 are formed, may be separated into each gas sensor. Similarly, a lubricant layer 23 is formed on the protective layer 35 on the glass substrate 80 to manufacture the magnetic disc 11.

In the fifth embodiment, the lamination structure on the substrate 21 of the gas sensor 20 is the same as the lamination structure on the glass substrate 80 of the magnetic disc 11. Namely, the first electrode 22 includes a conductive layer made of the same magnetic material as that used in the soft magnetic layer 41 and the intermediate layer 42 of the magnetic disc 11. Therefore, the gas adsorption performance of the gas sensor 20 approaches that of the magnetic disc 11. Accordingly, it is possible to detect gas that adsorbs onto the magnetic disc 11 more precisely. It also becomes possible to detect corrosion or the like of a magnetic film.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present invention have been described in detail, it should be understood that the various changes, substitutions, and alterations could be made hereto without departing from the spirit and scope of the invention.

What is claimed is:

1. A magnetic disc apparatus comprising:
a magnetic recording medium disposed in a housing and having a first lubricant layer formed on a surface of the magnetic recording medium; and
a gas sensor disposed in the housing for detecting gas by adsorbing the gas on a detection surface of the gas sensor, the detection surface being formed with a second lubricant layer made of a same lubricant agent as a lubricant agent used for the first lubricant layer.

2. The magnetic disc apparatus according to claim 1, wherein:
a protective layer containing diamond like carbon is formed over the surface of the magnetic recording medium, and the first lubricant layer is formed over the protective layer; and
a film containing diamond like carbon is formed on the detection surface of the gas sensor, and the second lubricant layer is formed on the film containing diamond like carbon.

3. The magnetic disc apparatus according to claim 1, wherein a surface under the second lubricant layer is subjected to a surface roughing process.

4. The magnetic disc apparatus according to claim 1, wherein:
the gas sensor comprises:
a substrate made of piezoelectric material; and
electrodes formed on both sides of the substrate, and wherein the second lubricant layer is formed on at least one of the electrodes.

5. The magnetic disc apparatus according to claim 4, wherein:
the magnetic recording medium comprises a substrate of non-magnetic material and a magnetic layer formed on the substrate; and
one of the electrodes covered with the second lubricant layer comprises a layer made of a same magnetic material as a magnetic material of the magnetic layer.

6. A gas sensor comprising:
a substrate made of piezoelectric material;
a first electrode formed over a first surface of the substrate;
a second electrode formed on a second surface of the substrate other than the first surface; and
a lubricant layer made of a lubricant agent and formed on the first electrode.

7. The gas sensor according to claim 6, wherein a film containing diamond like carbon is disposed between the first electrode and the lubricant layer.

8. A manufacture method for a magnetic recording medium and a gas sensor, comprising:
disposing a first substrate made of non-magnetic material and a second substrate made of piezoelectric material in a same film forming chamber;
forming a magnetic layer over the first substrate and forming a magnetic layer made of a same magnetic material as a magnetic material of the magnetic layer and used as an electrode, respectively in the film forming chamber;
forming a protective layer made of non-magnetic material on the magnetic layer and forming a protective layer made of a same non-magnetic material as the non-magnetic material of the protective layer on the electrode, respectively in the film forming chamber;
unloading the first substrate formed with the protective layer and the second substrate formed with the protective layer, respectively from the film forming chamber;
forming a lubricant layer on the protective layer of the first substrate; and
forming a lubricant layer made of a same lubricant agent as a lubricant agent of the lubricant layer formed on the first substrate, on the protective layer of the second substrate.
